(19) United States
(12) Patent Application Publication
(10) Pub. No.: US 2004/0002738 A1
(43) Pub. Date: Jan. 1, 2004

(54) Implantable cardioverter defibrillator with automatic adjustment of shock energy content

(75) Inventors: Magnus Ohman, Hasselby (SE); Berit Larsson, Danderyd (SE)

Correspondence Address: SCHIFF HARDIN & WAITE, 6600 SEARS TOWER, 233 S WACKER DR, CHICAGO, IL, 60606-6473, US

(73) Assignee: St. Jude Medical AB

(21) Appl. No.: 10/437,110

(22) Filed: May 13, 2003

(30) Foreign Application Priority Data
Jun. 28, 2002 (SE) 0202042-8

(51) Int. Cl. A61N 1/39

(52) U.S. Cl. 607/5

(57) ABSTRACT

In an implantable cardioverter defibrillator capable of reducing the required energy content of defibrillation and/or cardioversion pulses, the ventricular and/or atrial capture threshold is measured, based on the measured threshold values, the value settings for the energy contents of the defibrillation and/or cardioversion pulses is increased or reduced in correspondence with variations in the measured capture thresholds.

### BACKGROUND OF THE INVENTION

1. Field of the Invention

The present invention generally relates to the field of implantable cardioverter defibrillators. More specifically, the invention relates to an implantable cardioverter defibrillator (ICD) of the type having a pulse generator for delivering pacing pulses to a chamber of a heart, a defibrillation unit for delivering cardioversion or defibrillation shocks to that chamber, sensing circuitry for sensing both intrinsic heart activity and capture following a delivered pacing pulse, and a control unit for controlling the timing and energy of the pacing pulses and the cardioversion and defibrillation shocks, respectively, and wherein the control unit determines capture threshold.

2. Description of the Prior Art

Heart defibrillation is currently performed by the discharge of a powerful voltage pulse between two electrodes. The electrodes are placed so the discharge takes place over the heart or a large part thereof. The energy in a pulse for heart or ventricular defibrillation typically amounts from a few joules up to a few dozen joules.

The high energy required in a defibrillation shock has shortcomings. For devices with both defibrillation and pacemaker functions, i.e. devices designed to normally operate as pacemakers, the high energy consumed from defibrillation shortens the life of the apparatus considerably. The powerful energy discharge also has certain adverse effects on the organism.

Thus, for implantable cardioverter defibrillators (ICD), there is a need for reducing the energy required for successively performing defibrillation or cardioversion of a human heart. In a modern ICD, cardioversion or defibrillation normally is performed by delivering one or more cardioversion or defibrillation shocks having a predetermined energy content. If the defibrillation or cardioversion is unsuccessful, then a second shock or a second series of shocks of a higher energy level is delivered. This procedure is repeated until the cardioversion or defibrillation is successful, or until the energy level reaches a preset or possible maximum level.

As readily understood, it is desired to break an arrhythmia or fibrillation using as shock energy that is as low as possible, and with as few shocks as possible, in order to conserve energy. Also, when an atrial fibrillation is to be terminated, the patient is normally conscious and therefore experiences a certain amount of pain for each shock. Thus, the discomfort for the patient increase with the energy content of the atrial shock and the number of shocks delivered.

Therefore, the first pulse or series of pulses, whether delivered for terminating arrhythmia or fibrillation in the atrium or the ventricle, should have as low energy content as possible, while still managing a successful defibrillation or cardioversion. Furthermore, in order to minimize patient risk, it is also desirable for the first pulse to have a high probability of success. Therefore, a defibrillation threshold (DFT) value is determined while the patient is sedated during implantation surgery. Then, ventricular fibrillation is induced and defibrillation shocks of varied energy content are delivered. The success of the defibrillation shocks are monitored and the surgeon sets the energy content of the first cardioversion and/or defibrillation shocks to a level where a high probability of success can be expected.

However, it is well known that the energy level required not only varies from one patient to another, or due to differences in lead configuration and electrode placement of an ICD in a specific patient. The required energy level may also vary over time for a specific ICD when in an implanted position in a specific patient, for instance due to effects of anti-arrhythmia medication. Therefore, there is a risk that the defibrillation threshold determined upon implantation of the ICD, and stored therein, differs from the actual defibrillation threshold at the time when cardioversion or defibrillation is required.

If the stored DFT value is greater than the actual or real defibrillation threshold, then the energy content of the first pulse would be higher than required. Even though successful, this would result in an unnecessarily high energy consumption of the ICD for that cardioversion or defibrillation.

On the other hand, if the stored DFT value is less than the actual defibrillation threshold, then there is an increased risk of the first pulse or the first series of pulses being unsuccessful. This would result in a second pulse or second series of pulses with an increased energy content being delivered. Thus, this would also result in an unnecessarily high energy consumption of the ICD for that particular cardioversion or defibrillation.

It is known in the art of cardiac stimulators to provide, during the normal operation of the cardiac stimulator after implantation surgery, ventricular capture threshold determination to maintain the energy of the stimulation pulses at a level just above that which is needed to effectuate capture, see e.g. PCT Application WO 99/65566. It is also known to provide automatic capture pacing in an ICD system, see e.g. U.S. Pat. No. 6,327,498. Furthermore, in a recent study where defibrillation threshold (DFT) was determined for a number of patients and compared to the ventricular capture threshold (VCT) for each patient, it was concluded that VCT for a heart is related to the DFT for that heart, see “Ventricular capture threshold correlates with ventricular defibrillation threshold”; Val-Mejias J. E., Kroll M. W., and Syed Z.; Europace Supplements, Vol. 2, June 2001, No. 743, page B41.

However, it is still not possible to determine the defibrillation threshold after the patient is out of implantation surgery, i.e. during the normal every-day operation of an ICD. This is mainly due to the fact that the risk of inducing ventricular fibrillation without the presence of external equipment and qualified personnel would be too great. Furthermore, the pain and discomfort for the patient would probably be unbearable.

### SUMMARY OF THE INVENTION

An object of the present invention is to provide an implantable cardioverter defibrillator in which the above stated drawbacks are significantly reduced.

This and other objects are achieved according to the present invention by providing an ICD of the type initially described wherein the energy content of defibrillation or cardioversion shocks in an ICD is adjusted dependent on a determined pacing or ventricular capture threshold (VCT).

In accordance with the present invention, the ventricular capture threshold is repeatedly determined and the increase and decrease thereof is monitored. This information is then used for adjusting the energy content of the defibrillation and/or cardioversion shocks, such that the overall energy consumption of successful defibrillations and/or cardioversions can be reduced while still maintaining the same probability for success. Thus, the present invention provides an ICD having the capability of adjusting the defibrillation or cardioversion energy level settings dependent on varying requirements or conditions of a patient during normal every-day operation of the ICD.

The basis for the adjustment of defibrillation and cardioversion energy is that a decrease in VCT would imply a decreased DFT, which would enable the ICD to deliver a defibrillation shock with a reduced energy content without increasing the risk of unsuccessful defibrillation. Similarly, an increase in the determined VCT would imply an increased DFT, which in turn would require a defibrillation shock with a higher energy content in order to maintain the same probability for a successful defibrillation. By adjusting the energy content of the defibrillation shock, the risk of having to deliver back-up shocks is clearly reduced. As can be readily understood, such an adjustment of the energy content, whether an increase or a decrease, can significantly reduce the energy consumption for the shocks required to terminate an arrhythmia, and thereby significantly prolong the life of the ICD batteries and the time between successive replacement surgeries.

In an embodiment of the invention, an initial defibrillation threshold is determined during implantation surgery and stored in the ICD. The initial energy content of a first defibrillation shock, or a first series of defibrillation or cardioversion shocks, is then set to an initial value related to the determined initial DFT, e.g. the initial DFT+safety margin. Also, an initial ventricular capture threshold (VCT) value is determined and stored in the ICD. In use, the ICD measures changes in the VCT and adjusts the energy content of defibrillation shocks and the energy content of cardioversion shocks dependent on the determined VCT. In practice, if the determined VCT shows an increase over the initial VCT, then the energy content of the cardioversion and defibrillation shocks are increased in relation to their initial value, and vice versa.

In a preferred embodiment of the invention, the setting of energy levels for defibrillation and/or cardioversion shocks is adjusted whenever the setting of pacing energy levels is adjusted. That is, whenever the determined VCT results in an increase of the energy level setting for the pacing pulses, the energy levels for defibrillation and/or cardioversion shocks is/are also set at a higher level. Preferably, the energy levels are adjusted in predetermined steps. Then, a change of one step for the pacing pulse energy level results in a change of one step in the defibrillation/cardioversion shock energy levels.

In an embodiment, the energy levels of the pacing pulses are adjusted by adjusting the pulse amplitude in steps of 0.2-0.5 volts with maintained pulse durations. In this embodiment, the settings of defibrillation shocks are adjusted in corresponding steps of 2-5 joules for following the adjustments in said pacing energy level steps of 0.2-0.5 volts.

The pacing energy levels instead, or also, can be modified by adjustment of the pacing pulse duration. Of course, the adjustment in the setting of the defibrillation shocks may be performed in correspondence with a change in the pacing energy setting, regardless of this change is performed by adjusting the amplitude or duration, or both, of the pacing pulses.

In another embodiment, the energy level settings for the defibrillation and cardioversion shock are adjusted at predetermined time intervals. Preferably, the setting is adjusted at least every two days, more preferably at least once a day, and even more preferably every 8 hours.

In a preferred embodiment of the invention, the ICD utilizes a single ventricular lead for delivering both defibrillation and cardioversion shocks, as well as for delivering the lower energy pacing pulses. Such an implantable lead is disclosed in U.S. Pat. No. 6,327,498, the teachings of which are incorporated herein by reference. This configuration also enables autocapture detection with the same lead.

In other embodiments, the defibrillation and cardioversion shocks may be delivered through one ventricular lead, and the pacing pulses delivered through another ventricular lead. Then, the lead for pacing preferably is also used for sensing ventricular capture.

Any defibrillation or cardioversion shock sequence can be used within the scope of the present invention. For instance, the cardioversion may be in accordance with a stepped cardioversion algorithm, such as disclosed in U.S. Pat. No. 5,620,469. Another example can be found in European Application 588 125, which discloses an apparatus for defibrillating a human heart using a sequence of combined pacing pulses and defibrillation shocks.

In further embodiments of the present invention, the ICD is arranged for terminating an arrhythmia or fibrillation of the atrium of the heart, either in addition to or instead of the ventricular arrhythmia terminating capabilities described above in relation to the present invention. In these embodiments, the energy content of the atrial defibrillation shock is adjusted dependent on determined ventricular or atrial capture threshold. Thus, and as explained above, in addition to the energy preservation, the pain and discomfort for a patient may be significantly reduced in comparison to the use of conventional atrial defibrillators.

In use, when the ventricular capture threshold provides the basis for adjusting the energy content of the atrial defibrillation shock, the adjustment may be performed in the same manner as described above for the adjustment of the ventricular defibrillation shock. This is also the case when an atrial capture threshold provides the basis for adjusting the energy content of an atrial defibrillation shock. Then, however, instead of determining ventricular capture threshold, an atrial capture threshold is determined which forms the basis for the changes in the energy content of the atrial defibrillation shocks. In both cases, the atrial pacing pulses and defibrillation shocks are delivered through a trial respective leads or a single atrial lead.

Once the capture threshold has been determined and the defibrillation energy levels has been adjusted accordingly, use may be made of means and methods and energy levels known to those skilled in the art, and therefore needing no further description herein, for terminating ventricular or atrial arrhythmia, fibrillation or flutter.

### DESCRIPTION OF THE DRAWINGS

FIG. 1 is a diagrammatic, perspective view of an ICD system according to the present invention.

FIGS. 2 and 3 are schematic illustrations of an ICD according to alternative embodiments of the present invention.

FIG. 4 illustrates in diagrammatic form changes in defibrillation energy settings in accordance with the present invention.

### DESCRIPTION OF THE PREFERRED EMBODIMENTS

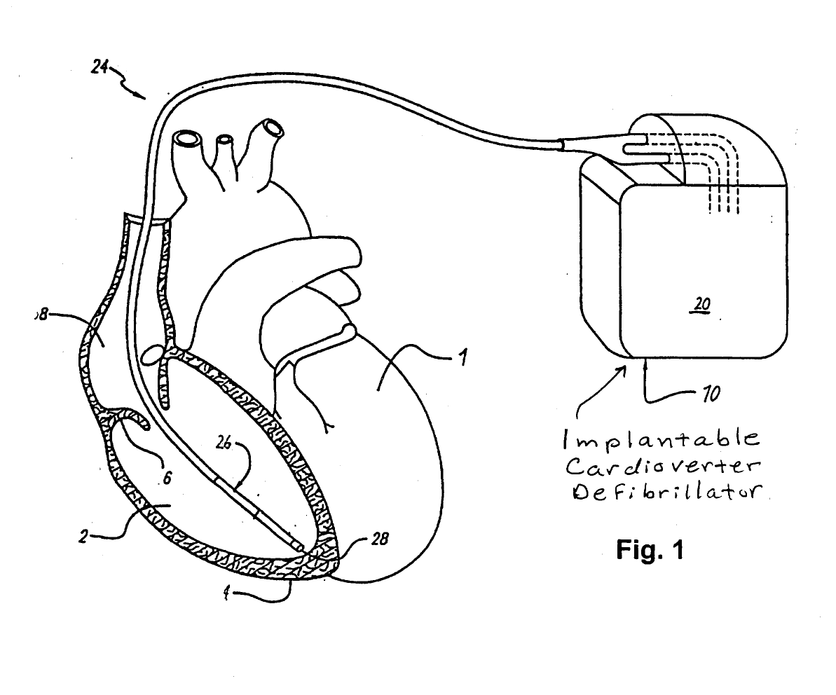

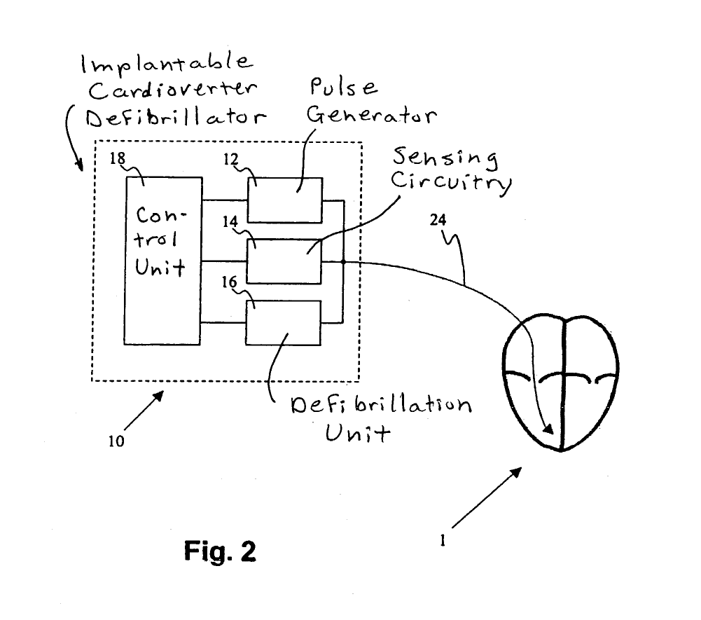

With reference first to FIGS. 1 and 2, a first embodiment of an implantable defibrillation system of the present invention is generally shown. The system includes an implantable cardioverter defibrillator (ICD) 10, typically subcutaneously implanted between the skin and the ribs of the patient. An implantable, ventricular ICD lead system 24 is passed through a vein into the right ventricle 2 of a heart 1. The distal end of the lead system 24 has a tip electrode 28 contacting the interior of the ventricle, preferably at its apex 4.

According to the described embodiment, an elongated, annular shocking coil electrode 26, also referred to as a ring electrode, is spaced at a distance of about 1.5-3.0 cm from the tip electrode 28. The shocking coil extends in a direction towards the region of the tricuspid valve 6 between the right atrium 8 and the right ventricle 2 and typically has a length of about 2-6 cm.

Each of these electrodes is connected, via the ventricular lead 24, to the circuitry contained in the ICD 10. The metallic enclosure or “can” of the ICD 10 also forms an electrode surface 20.

Although a variety of lead configurations can be used to pace the heart, to sense the intrinsic depolarizations of the heart, and to deliver defibrillation or cardioversion shocks to the heart, according to a first embodiment of the invention ventricular pacing and sensing is accomplished using the tip electrode 28 and the shocking coil electrode 26 of the ventricular lead 24. Defibrillation is delivered using the shocking coil electrode 26 and the can electrode 20. Thus, the ventricular lead 24 may be utilized both for ventricular pacing and as a defibrillator lead. According to a second embodiment, however, both ventricular pacing and defibrillation are delivered using the shocking coil electrode 26 and the can electrode 20.

In a further embodiment, the lead has two ring electrodes as well as a tip electrode, for instance as described in the above-mentioned U.S. Pat. No. 6,327,498, which is incorporated herein by reference. Then, defibrillation is delivered using one ring electrode and the can, and pacing is delivered using the other ring electrode and the can.

The ICD 10 has a can 20, as mentioned above, which also functions as an electrode. The can 20 contains a pulse generator 12 for delivering pacing pulses, sensing circuitry 14 for detecting ventricular evoked response or capture, and a defibrillation unit 16 for delivering cardioversion and/or defibrillation shocks. The ventricular lead 24 is connected to each of these units via a header attached to the can 20. Furthermore, the can 20 contains a control unit 18 arranged for receiving and processing sensing information from the sensing circuitry 14, and for controlling the pulse generator 12 and the defibrillation unit 16, thereby also controlling the timing and delivery of pacing pulses, cardioversion shocks and defibrillation shocks to the heart.

For the purposes of the present invention, use can be made of sensing circuitry, pulse generators and defibrillation and cardioversion units that are known. Since the basics and functioning of such elements are familiar to the person skilled in the art, they will not be described in further detail herein.

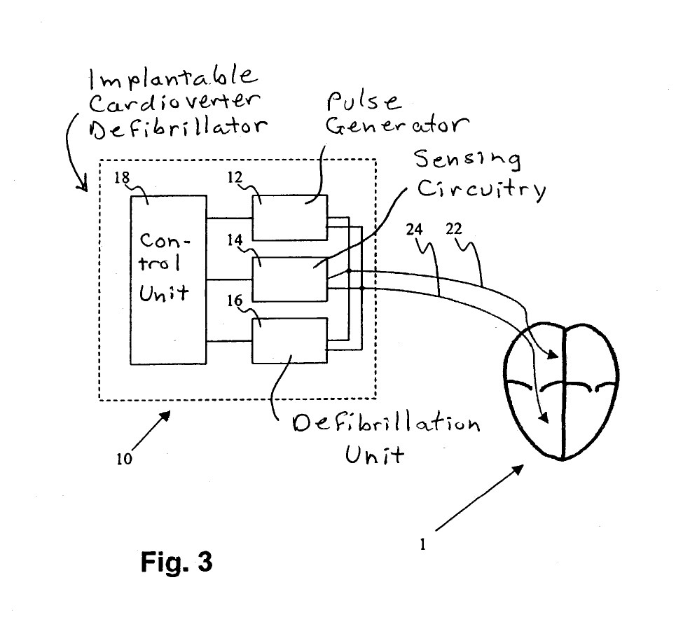

FIG. 3 shows an alternative embodiment of the present invention. In this embodiment, the ICD system also has an atrial lead 22, which has a similar configuration as the ventricular lead 24 described above. Thus, the atrial lead 25 also has a tip electrode and a ring electrode spaced apart from the tip electrode. The tip electrode of the atrial lead 22 is positioned in the atrium of the heart 1, as is schematically depicted in FIG. 3.

Furthermore, as with the ventricular lead 24, the atrial lead 22 is also connected to the pacing, sensing and defibrillation units via the header of the ICD 10, wherein the sensing circuitry 12 is further arranged for sensing atrial capture, the pulse generator 14 is further arranged for delivering atrial pacing pulses, and the defibrillation unit 16 is further arranged for delivering atrial defibrillation shocks.

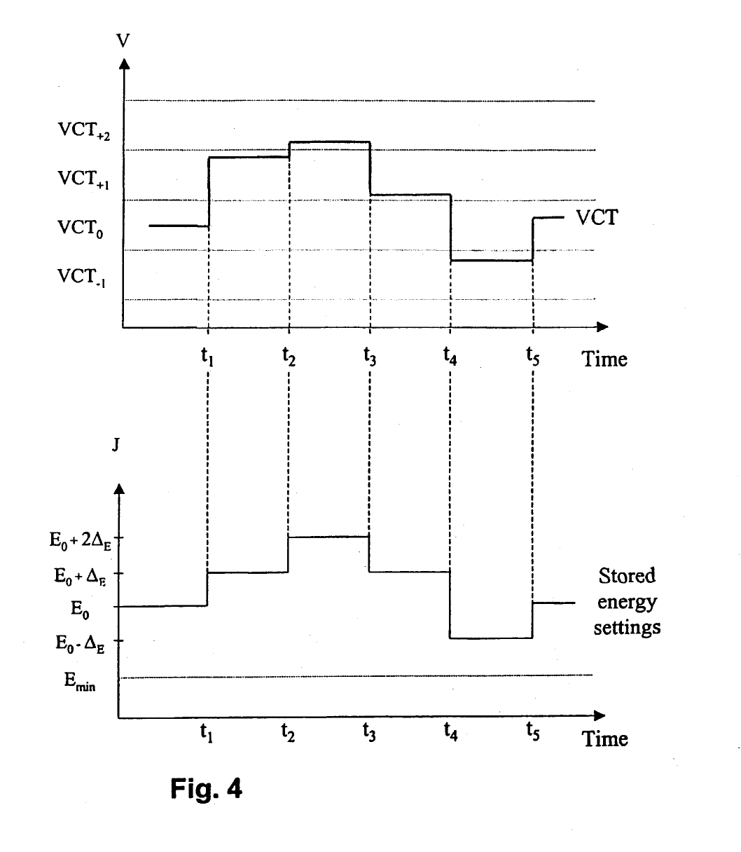

FIG. 4 shows in diagrammatic form the changes in the ventricular capture threshold (VCT) and the corresponding changes in the setting of the energy content for the first ventricular defibrillation shock. First, it should be noted that ventricular defibrillation or cardioversion therapy delivered by the ICD 10 can be in the form of a single defibrillation shock, or as a series of defibrillation or cardioversion shocks delivered to the ventricle of the heart, using the ventricular shocking coil electrode 26 and the can electrode 20. Thus, when a single defibrillation shock is mentioned in the following description, it may be exchanged for a series of defibrillation shocks, or a series of cardioversion shocks.

As mentioned above, the initial defibrillation threshold (DFT) is determined upon implantation of the ICD 10 in a patient. Then, the initial defibrillation energy, i.e. the energy content of the first defibrillation shock, is set to an initial value E₀ that exceeds the determined DFT by a selected safety margin. For obvious reasons, the value of E₀ depends on the determined DFT and may vary greatly from patient to patient. However, a typical value for E₀ lies within the range of 5-15 joules.

A minimum value Eₘᵢₙ for the stored defibrillation energy may also be determined. This value can correspond to E₀, or alternatively can be a value less than E₀ if the initial safety margin is set at a sufficiently high level.

Also, an initial ventricular capture threshold (VCT) is determined and stored in the control unit 18 of the ICD 10. This initial VCT is denoted V₀ in the diagram of FIG. 4. As regards the methods for determining DFT and for determining VCT, use can be made of conventional methods well known to those skilled in the art, which therefore need not be described in further detail herein.

Following completion of the implantation, i.e. during the normal running operation of the implanted ICD 10, the control unit 18 initiates the determination of the VCT value at regular intervals. Then, the setting of the energy content of pacing pulses is regulated to meet the detected changes in the determined VCT. This is well known within the art of cardiac stimulators and is normally performed by adjusting the voltage of the pacing pulse.

However, according to the present invention, the determined changes in the VCT are also used as a basis for adapting the energy content of the defibrillation and/or cardioversion shocks. This is illustrated in FIG. 4, wherein it is depicted how the VCT level changes over time at selected measurement intervals. In reality, the ventricular capture threshold may change continuously over time. The points in time t₀ through t₅, as shown in FIG. 4, simply denote when it is intended that the setting of the energy content of a defibrillation shock is to be amended in adaptation to a determined new VCT value.

As illustrated by the diagram of FIG. 4, each determined VCT value is determined to lay within one of several VCT value ranges, which are denoted as VCT₋₁ through VCT₊₂ in the diagram, where VCT₀ is the initial VCT value. A determined change in VCT value from one range to the next results in a change in the setting of the energy content in the pacing pulse by one step, which typically corresponds to a 0.2 to 0.5 V change in the voltage of the pacing pulse.

Furthermore, the setting of the energy content E of the defibrillation shock is also incremented or decremented by a single step for each determined change in VCT value from one range to the next. This is clearly illustrated in FIG. 4, wherein a determined change in the VCT value from range VCT₀ to VCT₊₁, at time t₁ results in a change in the defibrillation energy setting from E₀ to E₀+Δ_{E}. It should be noted that for simplicity of description, only four ranges have been illustrated. However, the preferred number of VCT value ranges are selected in accordance with the desired number of different settings of defibrillation energy content values, which probably will be more than four.

Furthermore, a further increase in the determined VCT value at time t₂, such that the detected VCT value is in the new range VCT₊₂, results in a corresponding increase in the defibrillation energy setting to E₀+2Δ_{E}. Likewise, at time t₃, a detected decrease of the VCT value results in the defibrillation energy setting returning to E₀+Δ_{E}. At t₄, a further detected decrease in the VCT value to within the range VCT₋₁ results in a corresponding decrease in the defibrillation energy setting to E₀−Δ_{E}. As can be seen in the figure, E₀−Δ_{E} is still above the set minimum value Eₘᵢₙ for the energy content of the defibrillation pulse. If the minimum value of Eₘᵢₙ would have been more than E₀−Δ_{E}, then the new value for the defibrillation energy setting should have been set to E₀ or Eₘᵢₙ.

In preferred embodiments of the invention, the value of each step Δ_{E} for setting the defibrillation energy content is between 2 and 5 Joules. For patients where E₀ is set to about 5 Joules or lower, then Δ_{E} may preferably be selected to lie within the range of 1 to 2 joules.

According to the described preferred embodiment, the illustrated change in the energy content setting is performed every 8 hours, i.e. the difference in time between TN and tₙ₊₁ is 8 hours. It is to be noted that although the ventricular capture threshold according to the present embodiment is determined every 8 hours for use as a basis for adapting defibrillation energy settings, VCT can be determined at a higher frequency for use as a basis for adapting pacing energy settings. However, according to this preferred embodiment, such modifications of the pacing energy are scheduled to be performed simultaneously with the modifications of the defibrillation energy settings.

As will be appreciated by those skilled in the art, the present invention is in no way restricted to any particular choice of time periods between defibrillation energy settings and or pacing energy settings. Furthermore, whenever a loss of capture for a delivered pacing pulse is detected, the energy content of the pacing pulse is increased until capture is detected. Thereafter, The ICD of the present embodiment performs a measurement of the VCT in order to enable a reduction of the pacing energy setting to a suitable value.

It should be noted that the above described embodiment of the present invention is operable with the ICD system of FIGS. 1 and 2, as well as with the ICD system of FIG. 3, i.e. with a ventricular and an atrial lead. In both cases, the ventricular capture threshold is determined by using detection of evoked responses sensed by the ventricular lead.

In further embodiments of the invention, and with reference to FIG. 3, the ICD is arranged for delivering atrial defibrillation pulses to the atrium of a human heart.

In one embodiment, the energy content of atrial defibrillation pulses are set on the basis of the determined ventricular capture threshold referred to above. In this embodiment, the atrial lead 22 is used for delivering the atrial defibrillation pulses to the atrium of the heart. The same atrial lead may also be used for sensing atrial evoked response and for delivering atrial pacing pulses. Furthermore, the atrial defibrillation energy level settings are adjusted in a corresponding manner to that described above for the adjustment of ventricular defibrillation energy level setting illustrated in FIG. 4, In the case where both atrial and ventricular defibrillation pulse settings are adjusted in accordance with variations in the VCT, these adjustments preferably are performed simultaneously, i.e. whenever the ventricular defibrillation settings are adjusted, so also are the atrial defibrillation settings.

Thus, according to this embodiment, the ventricular capture threshold is determined on a regular basis, and the energy content setting for atrial defibrillation and/or cardioversion is adjusted in correspondence with determined changes in the ventricular capture threshold, i.e. in the same manner as in the above-described method of adjusting the ventricular defibrillation energy settings. Therefore, reference is made to the above description of ventricular defibrillation energy content adjustment for the detailed description of how the atrial defibrillation energy settings are adjusted in accordance with determined atrial defibrillation threshold variations.

Furthermore, in another example of the embodiments where the ICD is arranged for delivering atrial defibrillation pulses to the atrium of a human heart, the energy content of atrial defibrillation pulses are set based on determined atrial capture threshold instead of determined ventricular capture threshold. The principle for adjusting the atrial defibrillation pulse settings is the same as described above for ventricular defibrillation and VCT. In this embodiment, an atrial lead, such as the atrial lead 22 shown in FIG. 3, is used both for sensing atrial-evoked response and for delivering atrial pacing and defibrillation pulses. The general principles of the present invention, as illustrated by the diagrams of FIG. 4, are equally applicable for this embodiment. According to this embodiment, the atrial capture threshold is determined on a regular basis, and the energy content setting for atrial defibrillation and/or cardioversion is adjusted in correspondence with determined changes in the atrial capture threshold, i.e. in the same manner as in the above described method of adjusting the ventricular defibrillation energy settings. Therefore, reference is again made to the above description of ventricular defibrillation energy content adjustment for the detailed description of how the atrial defibrillation energy settings are adjusted, in this case in accordance with determined atrial defibrillation threshold variations.

It should be noted that the above described arrangement for adjusting the ventricular defibrillation energy settings dependent on the determined ventricular capture threshold, as well as the arrangement for adjusting the atrial defibrillation energy settings dependent on determined ventricular or atrial capture threshold, may be combined in a single ICD, such as the ICD described with reference to FIG. 3.

Although modifications and changes may be suggested by those skilled in the art, it is the intention of the inventors to embody within the patent warranted hereon all changes and modifications as reasonably and properly come within the scope of their contribution to the art.

We claim as our invention:

1. An implantable cardioverter defibrillator comprising:
a pulse generator adapted to interact with at least one chamber of a heart for delivering pacing pulses, having an energy content, to said at lest one chamber;
a defibrillation unit adapted for interaction with said at least one chamber for delivering at least one shock, selected from the group consisting of a cardioversion shock and a defibrillation shock, to said at least one chamber;
sensing circuitry adapted for interact with said at least one chamber for sensing both intrinsic cardiac activity and capture following a pacing pulse delivered by said pulse generator; and
a control unit connected to said pulse generator for controlling an energy content of said pacing pulses, and connected to said sensing circuitry for receiving a signal therefrom from which said control unit determines a capture threshold, and connected to said defibrillation unit for adjusting an energy content of said shock dependent on the capture threshold determined by said control unit.

2. An implantable cardioverter defibrillator as claimed in claim 1 wherein said control unit increases said energy content of said shock dependent on an increase in said capture threshold, and decreases said energy content of said shock dependent on a decrease in said capture threshold.

3. An implantable cardioverter defibrillator as claimed in claim 1 wherein said control unit adjusts said energy content of said shock whenever a change in said capture threshold is detected by said control unit.

4. An implantable cardioverter defibrillator as claimed in claim 1 wherein said control unit adjusts said energy content of said shock dependent on said capture threshold at selected time intervals.

5. An implantable cardioverter defibrillator as claimed in claim 1 wherein said control unit also adjusts said energy content of said pacing pulses dependent on said capture threshold, and wherein said control unit adjusts the energy content of said shock whenever said control unit adjusts the energy content of said pacing pulses.

6. An implantable cardioverter defibrillator as claimed in claim 1 wherein said shock is a cardioversion shock, and wherein said control unit operates said defibrillation unit to deliver a plurality of cardioversion shocks, respectively having an energy content which changes in steps from shock to shock in accordance with an algorithm for stepped cardioversion, and wherein said control unit adjusts the respective energy content of said cardioversion shocks dependent on said capture threshold.

7. An implantable cardioverter defibrillator as claimed in claim 1 wherein said defibrillation unit includes a ventricular lead adapted for placement in the ventricle of the heart for delivering said shock from said defibrillation unit to the ventricle, and wherein said pulse generator and said sensing circuitry include a ventricular lead adapted for placement in the ventricle of the heart for delivering said pacing pulses to the ventricle and for providing said signal to said control unit, said control unit determining a ventricular capture threshold, as said capture threshold, from said signal.

8. An implantable cardioverter defibrillator as claimed in claim 7 wherein said defibrillation unit, and said pulse generator and said sensing circuitry, share a single ventricular lead.

9. An implantable cardioverter defibrillator as claimed in claim 7 wherein said defibrillation unit includes an atrial lead adapted for placement in the atrium of the heart for delivering at least one shock to the atrium, and wherein said pulse generator includes an atrial lead adapted for placement in the atrium of the heart for delivering said pacing pulses to the atrium, and wherein said control unit adjusts the energy content of the shock delivered to the atrium and the energy content of the shock delivered to the ventricle dependent on said ventricular capture threshold.

10. An implantable cardioverter defibrillator as claimed in claim 9 wherein said defibrillation unit and said pulse generator share a single atrial lead.

11. An implantable cardioverter defibrillator as claimed in claim 1 wherein said defibrillation unit includes an atrial lead adapted for placement in the atrium of the heart for delivering at least one shock to the atrium.

12. An implantable cardioverter defibrillator as claimed in claim 11 wherein said pulse generator is also connected to said atrial lead, and wherein said atrial lead delivers said pacing pulses to the atrium.

13. An implantable cardioverter defibrillator as claimed in claim 12 wherein said sensing circuitry is connected to said atrial lead and wherein said control unit determines an atrial capture threshold, as said capture threshold, from said signal from said sensing circuitry.

14. An implantable cardioverter defibrillator as claimed in claim 11 further comprising a ventricular lead connected to said sensing circuitry, and wherein said control unit determines a ventricular capture threshold, as said capture threshold, from said signal from said sensing circuitry.

15. An implantable cardioverter defibrillator as claimed in claim 1 wherein said control unit adjusts an energy content of said pacing pulses dependent on said capture threshold at selected time intervals.
